SEQUENCE LISTING (6 sequences)

SEQ ID NO: 1 - DNA, 20 bp, Artificial Sequence
  Description of Artificial Sequence SSP-Primer T
gccgaagggcatgagctgca

SEQ ID NO: 2 - DNA, 20 bp, Artificial Sequence
  Description of Artificial Sequence SSP-Primer C
gccgaagggcatgagctgcg

SEQ ID NO: 3 - DNA, 18 bp, Artificial Sequence
  Description of Artificial Sequence PCR Optimizer
cgtgatgagctgcacggt

SEQ ID NO: 4 - DNA, 22 bp, Artificial Sequence
  Description of Artificial Sequence 1stPCR-Primer1
cacaaagaattccagagaaata

SEQ ID NO: 5 - DNA, 17 bp, Artificial Sequence
  Description of Artificial Sequence 1stPCR-Primer2
gtaaacgtccctgtgct

SEQ ID NO: 6 - DNA, 648 bp, Homo sapiens
gtaaacgtccctgtgctaggtcttttgcaggcacagcttttcctccatgagtacgtattttgaaactcaagatcgcattcatgcgtcttcacctggaaggggtccatgtgcccctccttctggccaccatgcgaagccacactgacgtgcctctccctccctccaggaagcctacgtgatggccagcgtggacaacccccacgtgtgccgcctgctgggcatctgcctcacctccaccgtgcagctcatcaygcagctcatgcccttcggctgcctcctggactatgtccgggaacacaaagacaatattggctcccagtacctgctcaactggtgtgtgcagatcgcaaaggtaatcagggaagggagatacggggaggggagataaggagccaggatcctcacatgcggtctgcgctcctgggatagcaagagtttgccatggggatatgtgtgtgcgtgcatgcagcacacacacattcctttattttggattcaatcaagttgatcttcttgtgcacaaatcagtgcctgtcccatctgcatgtggaaactctcatcaatcagctacctttgaagaattttctctttattgagtgctcagtgtggtctgatgtctctgttcttatttctctggaattctttgtg

(19) United States
(12) Patent Application Publication
(10) Pub. No.: US 2010/0261186 A1
(43) Pub. Date: Oct. 14, 2010

(54) POLYMORPHISM IDENTIFICATION METHOD

(75) Inventor: Tetsuya TANABE, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS CORPORATION, Tokyo (JP)

(21) Appl. No.: 12/756,492

(22) Filed: Apr. 8, 2010

(30) Foreign Application Priority Data
Apr. 10, 2009 (JP) P2009-095913

(51) Int. Cl. C12Q 1/68

(52) U.S. Cl. 435/6

(57) ABSTRACT

The present invention is to provide a method for identifying a polymorphism with high sensitivity and high accuracy. The method of the present invention includes: performing a nucleic acid chain extension reaction and identifying the polymorphism of the nucleic acid contained in a test nucleic acid sample. The extension reaction is conducted with use of: a nucleic acid in a test nucleic acid sample as a template, a type I detection primer which hybridizes with a region including the polymorphic site of a nucleic acid whose polymorphic site nucleotide sequence consists of a first nucleotide sequence, and a polymerase having no strand displacement activity. The reaction is conducted with the presence of an inhibitory oligonucleotide which contains a nucleotide sequence complementary to the sequence of a region including the polymorphic site of a nucleic acid whose polymorphic site nucleotide sequence consisting of a second nucleotide sequence.

### BACKGROUND OF THE INVENTION

1. Field of the Invention

The present invention relates to a method for identifying a polymorphism more sensitively and more accurately than prior art methods.

2. Description of Related Art

With the recent progress in genetic engineering technologies and gene recombination technologies, genetic tests through nucleic acid analyses have been widely used in applications to medical services, researches, and industries. Such tests are to detect the presence of DNA which has a target nucleotide sequence within a sample, and have been applied not only to diagnosis and treatment of diseases, but also to food inspection and other various fields. In particular, a genetic polymorphism such as a SNP (Single Nucleotide Polymorphism) is considered to be a major factor contributing to the individual difference in the vulnerability against a specific disease such as cancer, the drug metabolizing capacity, and so forth. Genetic polymorphism analyses have been widely conducted not only in academic researches but also in actual clinical tests. Therefore, highly accurate and quick methods for detecting a genetic polymorphism have been enthusiastically developed.

As to the method for detecting and identifying a genetic polymorphism, there are many reported methods in which artificially synthesized polynucleotides such as probes and primers are used to examine the nucleotide sequences of nucleic acids. For example, some methods are to analyze the nucleotide sequence of a SNP serving as the analysis target and its neighboring region by molecular-biological enzymatic reactions. Such methods can be exemplified by: a method in which a region including a polymorphism such as a SNP can be detected by PCR (Polymerase Chain Reaction) amplification; and a method in which a SNP can be detected by a ligation reaction using a probe including the detection target SNP at the 3′ end and a probe including a nucleotide adjacent to the 5′ side of the SNP, at the 5′ end, and subsequent determination regarding the obtainability of a polynucleotide bound with these two probes.

In particular, often employed SNP analysis methods are SSP-PCR (Sequence Specific Primer-PCR) method and ASP-PCR (Allele Specific Primer-PCR) method, in which a SNP can be detected by PCR using a primer specifically bindable to a specific nucleotide sequence, allele, and the like, and subsequent determination regarding the presence/absence of the PCR product. The reason is that, since the detection and recognition of a nucleotide sequence (genetic polymorphism) can be carried out concurrently with enhancement of its signal, the polymorphism detection by means of the SSP/ASP-PCR method can enable the SNP detection even in the case where only a small amount of specimen is available, or the case where the nucleic acid concentration in a sample is very low, like a case of a specimen in a clinical test, and therefore these methods are very useful.

On the other hand, the SSP-PCR method and the ASP-PCR method involve a problem in that, due to a low flexibility in the design of the primer nucleotide sequence, sufficient sensitivity may not be achieved depending on the nucleotide sequence of the target genetic polymorphism to identify. Therefore, various methods have been disclosed in order to improve the identification accuracy upon identification of a genetic polymorphism such as a SNP with use of the SSP-PCR method or the ASP-PCR method. For example, there is a disclosed method (1) for detecting the presence/absence of a specific known nucleic acid sequence, wherein the method comprises: adding a primer complementary to the specific known sequence and a competitor oligonucleotide primer having nucleotide mismatch(es) with respect to the known sequence, to a nucleic acid or nucleic acid mixture sample so as to produce a competitive condition; selectively hybridizing between the specific known sequence and the primer substantially complementary thereto under the competitive condition; extending the selectively hybridized primer from its 3′ end so as to synthesize an extension product which is complementary to the nucleotide strand hybridized with the primer; and identifying the extension product (for example, refer to Patent Document 1). The method is to improve the identification accuracy for a nucleic acid including the known sequence by adding two types of primers which can hybridize with a common region among nucleic acids including the known sequence serving as the identification target, and utilizing competitive reactions thereof.

In addition, there is also a disclosed method (2) for identifying whether or not a nucleic acid having a polymorphism sequence site has a desired nucleotide sequence in the polymorphism sequence site, the method comprises: hybridizing an identification primer (here, this identification primer has on its 3′ end a nucleotide sequence for identifying the polymorphism sequence), and an oligonucleotide having no primer function (here, this oligonucleotide is partially or completely complementary to a region of the target nucleic acid on the 5′ side of the target nucleic acid), with a target nucleic acid having a polymorphism sequence site; and placing them in a reaction condition which allows the progress of the primer strand displacement extension reaction (for example, refer to Patent Document 2). The method is to improve the accuracy on whether or not the extension reaction has progressed, by using an oligonucleotide having no primer function at a downstream of the identification primer. If the identification primer is completely complementary to the target nucleic acid, the primer extension reaction can make progress by pushing out the oligonucleotide having no primer function and binding to the downstream of the identification primer; however, if a mismatch exists between the identification primer and the target nucleic acid, the primer extension reaction can hardly occur.

### REFERENCES

### Patent Documents

Patent Document 1: Japanese Patent (Granted) Publication No. 2760553

Patent Document 2: PCT International Publication No. WO 04/022743 pamphlet

### SUMMARY OF THE INVENTION

In the method (1), it is made easier for the nucleic acid including the known sequence as the identification target to hybridize to the primer which is complementary to the known sequence, than to the competitor primer, because nucleotide mismatch(es) is(are) included in the competitor primer. However, it may be difficult for these primers to be replaced with each other depending on their nucleotide sequences, and therefore the identification sensitivity may be insufficient.

On the other hand, the method (2) employs the strand displacement extension reaction, where the inhibitory effect on nonspecific reaction is insufficient. In particular, regarding a somatic mutation, a majority of nucleic acids contained in a sample are derived from normal genes, while mutated gene-derived nucleic acids account for a very small proportion. For this reason, much higher accuracy is required as compared to the case for detecting/identifying a SNP. However, it is difficult for the above-mentioned method (2) to detect such a somatic mutation. In addition, since the polymorphism detection site for detecting a SNP or such a polymorphic site is limited to the 3′ end of the primer, the flexibility in the design of the primer nucleotide sequence is very low, which is also problematic.

It is an object of the present invention to provide a method for identifying a polymorphism more sensitively and more accurately with use of the SSP-PCR method or the ASP-PCR method.

In view of the above-mentioned problems, the inventors of the present invention have conducted intensive studies. As a result, they have discovered that a nonspecific-nucleic acid extension reaction can be inhibited and thereby the polymorphism identification accuracy can be improved in the SSP-PCR method or the ASP-PCR method, by adding an oligonucleotide into a reaction solution when performing a nucleic acid extension reaction to cause hybridization between a template nucleic acid and a primer which can specifically hybridize with a certain type of polymorphic allele, wherein the oligonucleotide can hybridize with the template nucleic acid while overlapping with the 3′ end of the primer. This has led to the completion of the present invention.

That is, the present invention provides:

(1) a polymorphism identification method for identifying a polymorphism of a polymorphic site-containing nucleic acid, comprising:

(a) performing a nucleic acid chain extension reaction with use of a nucleic acid in a test nucleic acid sample as a template, a type I detection primer, and a polymerase having no strand displacement activity, with the presence of an inhibitory oligonucleotide, the type I detection primer being a primer which can hybridize with a nucleic acid in a region including the polymorphic site thereof whose polymorphic site nucleotide sequence consisting of a first nucleotide sequence, and the inhibitory oligonucleotide being an oligonucleotide which comprises a nucleotide sequence complementary to the sequence of a region including the polymorphic site of a nucleic acid whose polymorphic site nucleotide sequence consisting of a second nucleotide sequence; and

(b) identifying the polymorphism of the nucleic acid contained in the test nucleic acid sample, based on whether or not the type I detection primer has been extended in step (a);

wherein there is an overlap between the 5′ side of the region which can hybridize with the type I detection primer and the 3′ side of the region which can hybridize with the inhibitory oligonucleotide, within the nucleic acid whose polymorphic site nucleotide sequence consists of the first nucleotide sequence;

(2) the polymorphism identification method according to (1), wherein the 3′-end nucleotide of the inhibitory oligonucleotide is blocked so that the oligonucleotide has no function as a primer;

(3) the polymorphism identification method according to either (1) or (2), wherein the length of the overlap region between the 5′ side of the region which can hybridize with the type I detection primer and the 3′ side of the region which can hybridize with the inhibitory oligonucleotide, within the nucleic acid whose polymorphic site nucleotide sequence consists of the first nucleotide sequence, is five nucleotides or less;

(4) the polymorphism identification method according to either (1) or (2), wherein the length of the overlap region between the 5′ side of the region which can hybridize with the type I detection primer and the 3′ side of the region which can hybridize with the inhibitory oligonucleotide, within the nucleic acid whose polymorphic site nucleotide sequence consists of the first nucleotide sequence, is equal to or shorter than twice the length of the polymorphic site;

(5) the polymorphism identification method according to either (1) or (2), wherein the length of the overlap region between the 5′ side of the region which can hybridize with the type I detection primer and the 3′ side of the region which can hybridize with the inhibitory oligonucleotide, within the nucleic acid whose polymorphic site nucleotide sequence consists of the first nucleotide sequence, is two nucleotides or less;

(6) the polymorphism identification method according to any one of (1) through (5), wherein the polymorphism detection site of the type I detection primer to hybridize with the polymorphic site is located at its 3′ end;

(7) the polymorphism identification method according to any one of (1) through (6), wherein the nucleic acid chain extension reaction comprises

(i) denaturing the nucleic acid in the test nucleic acid sample into single strands;

(ii) annealing the single-stranded nucleic acid with the type I detection primer and/or the inhibitory oligonucleotide; and

(iii) extending the nucleic acid strand starting from the type I detection primer, wherein a Tm value of a hybrid between the inhibitory oligonucleotide and the nucleic acid whose polymorphic site nucleotide sequence consisting of the first nucleotide sequence, being higher than the temperature of (ii), and lower than the temperature of step (a);

(8) the polymorphism identification method according to any one of (1) through (6), wherein a thermodynamic stability of a hybrid between the inhibitory oligonucleotide and the nucleic acid whose polymorphic site nucleotide sequence consists of the first nucleotide sequence, is lower than that of a hybrid between the type I detection primer and the nucleic acid;

(9) the polymorphism identification method according to (7), wherein a cycle consisting of (i), (ii), and (iii) is repeated twice or more times in the nucleic acid chain extension reaction;

(10) the polymorphism identification method according to any one of (1) through (9), wherein the nucleic acid chain extension reaction is performed on a nucleic acid whose polymorphic site nucleotide sequence is different from the first nucleotide sequence, with the presence of a detection primer which can hybridize with the nucleic acid in a region including the polymorphic site thereof;

(11) the polymorphism identification method according to (10), wherein the detection primer is a type II detection primer which can hybridize with a nucleic acid in a region including the polymorphic site thereof whose polymorphic site nucleotide sequence consists of the second nucleotide sequence;

(12) the polymorphism identification method according to (11), wherein a thermodynamic stability of a hybrid between the inhibitory oligonucleotide and the nucleic acid whose polymorphic site nucleotide sequence consists of the first nucleotide sequence, is lower than that of a hybrid between the type I detection primer and the nucleic acid whose polymorphic site nucleotide sequence consists of the first nucleotide sequence, and a thermodynamic stability of a hybrid between the inhibitory oligonucleotide and the nucleic acid whose polymorphic site nucleotide sequence consists of the second nucleotide sequence, is lower than that of a hybrid between the type II detection primer and the nucleic acid whose polymorphic site nucleotide sequence consists of the second nucleotide sequence; and

(13) a polymorphism identification kit for use in the identification of a polymorphism of a polymorphic site-containing nucleic acid, comprising:

a type I detection primer which can hybridize with a nucleic acid in a region including the polymorphic site thereof whose nucleotide sequence consisting of a first nucleotide sequence; and

an inhibitory oligonucleotide which comprises a nucleotide sequence complementary to the sequence of a region including the polymorphic site of a nucleic acid whose polymorphic site nucleotide sequence consisting of a second nucleotide sequence,

wherein there is an overlap between the 5′ side of the region which can hybridize with the type I detection primer and the 3′ side of the region which can hybridize with the inhibitory oligonucleotide, within the nucleic acid whose polymorphic site nucleotide sequence consists of the first nucleotide sequence.

With use of the polymorphism identification method of the present invention, a nonspecific-nucleic acid extension reaction can be efficiently inhibited and thereby the polymorphism can be accurately and sensitively identified in the SSP-PCR method or the ASP-PCR method where the flexibility in the design of the polymorphism detection primer has been low so far. Particularly, it is possible to sensitively detect or identify a polymorphism such as a somatic mutation which has been so far difficult to detect or identify by conventional SSP-PCR or like method.

### BRIEF DESCRIPTION OF THE DRAWINGS

FIG. 1 is a schematic diagram showing a hybrid formed by hybridization between a type I nucleic acid and a type I detection primer, and a hybrid formed by hybridization between the type I nucleic acid and an inhibitory oligonucleotide.

FIG. 2 is a schematic diagram showing a state where the type I nucleic acid hybridizes with the type I detection primer and with the inhibitory oligonucleotide, and a state where a type II nucleic acid hybridizes with the type I detection primer and with the inhibitory oligonucleotide.

FIG. 3 shows the nucleotide sequence (SEQ. ID. No. 6) in the vicinity of the threonine-encoding codon at the amino acid position 790 in the EGFR gene.

FIG. 4 shows the consumption rate (K2%) of the mutant detection primer with variations of the content ratio of the mutant nucleic acid, measured in the example 1.

FIG. 5 shows the result of FCS measurement for respective test nucleic acid samples of the example 1 in scatter charts, wherein the y axis represents the consumption rate (K2%) of the mutant detection primer and the x axis represents the consumption rate (K2%) of the wild-type detection primer.

FIG. 6 shows the consumption rate (K2%) of the mutant detection primer with variations of the content ratio of the mutant nucleic acid, measured in the example 2.

### DETAILED DESCRIPTION OF THE INVENTION

The term “polymorphism” used in the present invention refers to an occurrence of variation in the nucleotide sequence of a same gene, between individuals within a certain group of biological species, or between cells within a same individual. Specifically, a specific nucleotide sequence of a gene derived from a certain cell may be different from a corresponding nucleotide sequence of the same gene derived from another individual of the same biological species, or another cell of the same individual, due to substitution, deletion, or insertion of one or a plurality of nucleotide(s); in which case, the gene is regarded to be polymorphic, and the site whose nucleotide sequence varies between both parties is referred to as the polymorphic site. The polymorphism may occur either in genomic DNA or in mitochondrial DNA.

The term “polymorphic site” used in the present invention refers to a site of a gene whose nucleotide sequence varies per each polymorphism. For example, assuming that in a certain kind of polymorphism, a first type (type I) nucleotide sequence is gggaaa and another non-type I (type II) nucleotide sequence is ggcaaa; the third nucleotide from the 5′ side, g in the type I and c in the type II, is regarded as the polymorphic site. In addition, the term “to identify a polymorphism” used in the present invention means to identify whether or not the polymorphic site nucleotide sequence of a nucleic acid contained in a test nucleic acid sample is the same as that of a certain kind of polymorphism. Accordingly, the nucleotide sequence of the polymorphism serving as the target of the polymorphism identification method of the present invention has to be elucidated to an extent which allows such identification.

The polymorphism serving as the identification target in the present invention is not specifically limited as long as the polymorphism satisfies the above-mentioned provisions and its nucleotide sequence has been elucidated to a detectable degree by a gene recombination or like technique. In addition, the polymorphism may be either inherent or acquired as often seen in a tumor or like cells. Examples of such a polymorphism can include a single nucleotide polymorphism (SNP), a microsatellite, and a somatic mutation. In the present invention, it is preferable to identify a kind of polymorphism in which one to five nucleotide(s) is(are) substituted, deleted, or inserted, and more preferably a kind of polymorphism in which one to three nucleotide(s) is(are) substituted, deleted, or inserted.

The test nucleic acid sample in the present invention is not specifically limited as long as the sample contains a nucleic acid which has a polymorphism to identify. The test nucleic acid sample may be a biological sample collected from an animal or the like, a sample prepared from a cultured cell lysate or the like, and a nucleic acid solution extracted and purified from a biological sample or the like. In particular, human-derived biological samples to be used for clinical or other tests and nucleic acid samples extracted and purified from such human-derived biological samples are preferred. Examples of such human-derived biological samples can include blood, bone marrow, lymph fluid, urea, sputum, ascites fluid, exudate fluid, amniotic fluid, peritoneal lavage fluid, lung lavage fluid, bronchial lavage fluid, bladder lavage fluid, pancreatic juice, saliva, semen, bile, and feces. In addition, the test nucleic acid sample may be directly used after the collection from an organism, or may be prepared before use. The preparation method is not specifically limited as long as DNA, RNA, or such a nucleic acid contained in the biological sample is not impaired, and a usual preparation method for biological samples can be applied. Besides, DNA extracted and purified from a biological sample and amplified by a PCR or like method, and cDNA synthesized from RNA contained in a biological sample with a reverse transcriptase may also be used. When DNA or the like extracted and purified from a biological sample is used, it is possible to amplify the polymorphic site-containing nucleic acid contained therein by PCR and to use the thus yielded amplification product as the test nucleic acid sample.

The polymorphism identification method of the present invention is a method to identify a polymorphism of a polymorphic site-containing nucleic acid, wherein the method comprises performing a nucleic acid chain extension reaction with use of a type I detection primer which can specifically hybridize with a type (type I) of a polymorphism, with the presence of an inhibitory oligonucleotide that specifically hybridizes with another type (type II) of the polymorphism.

The term “type I detection primer” used in the present invention refers to a primer which can hybridize with a region including the polymorphic site of a nucleic acid whose polymorphic site of the identification target polymorphism consists of a first nucleotide sequence (hereinunder, may be referred to as the “type I nucleic acid”). The type I detection primer may be any kind of primer which can hybridize with a partial region of the type I nucleic acid which contains the polymorphic site. The type I detection primer may be an oligonucleotide which comprises a nucleotide sequence completely complementary to the nucleotide sequence of the type I nucleic acid, or an oligonucleotide which comprises a nucleotide sequence complementary thereto except for one or several nucleotide mismatch(es). Preferably, the type I detection primer of the present invention is an oligonucleotide which comprises a nucleotide sequence completely complementary to the nucleotide sequence of the type I nucleic acid, as it can offer higher identification accuracy.

In addition, the type I detection primer may also have on its 5′ side an additional nucleotide sequence besides the region which can hybridize with the type I nucleic acid. Examples of such an additional sequence include a restriction enzyme recognition sequence and a sequence for labeling the nucleic acid.

Furthermore, the type I detection primer may be labeled so as to facilitate the detection of the extension product starting from the primer. The labeling substance is not specifically limited as long as it can be used for labeling nucleic acids. Examples thereof can include radioisotopes, fluorophores, chemiluminescent substances, and biotin.

It is preferable to design the type I detection primer so that its polymorphism detection site which can hybridize with the polymorphic site of the first (the type I) nucleic acid can be located at the 3′ side, rather than the 5′ side, of the primer. In particular, it is more preferable to design the type I detection primer so that its polymorphism detection site can be located within five nucleotides from the 3′ end of the primer, more preferably within two nucleotides from the 3′ end thereof, and particularly preferably at the 3′ end.

The term “inhibitory oligonucleotide” used in the present invention refers to an oligonucleotide which can hybridize with a region including the polymorphic site of a nucleic acid whose polymorphic site nucleotide sequence consists of a second nucleotide sequence (hereinunder, may be referred to as the “type II nucleic acid”), as well as being an oligonucleotide which comprises a nucleotide sequence complementary to the sequence of the region including the polymorphic site of the type II nucleic acid. Here, the second nucleotide sequence refers to another nucleotide sequence of the polymorphic site regarding the identification target polymorphism, which is different from the first nucleotide sequence. For example, if the identification target polymorphism is a SNP having two types of polymorphism, namely a wild-type and a mutant, the nucleotide sequence of the mutant can be regarded as the first nucleotide sequence and the nucleotide sequence of the wild-type can be regarded as the second nucleotide sequence. In addition, if the identification target polymorphism is a somatic mutation, the nucleotide sequence of the mutant can be regarded as the first nucleotide sequence and the nucleotide sequence of the normal type can be regarded as the second nucleotide sequence. If the polymorphism has three types of polymorphic site nucleotide sequences, either type of the nucleotide sequences other than the first nucleotide sequence can be selected as the second nucleotide sequence.

The type I detection primer can hybridize not only with the type I nucleic acid but also with the type II nucleic acid, under a low-temperature environment including the annealing step of the nucleic acid chain extension reaction. In this manner, the type I detection primer does hybridize with the type II nucleic acid, and the nonspecific-nucleic acid chain extension reaction does occur. This leads to a worsening of the polymorphism identification accuracy.

The polymorphism identification method of the present invention employs the inhibitory oligonucleotide which can specifically hybridize with a non-type I nucleic acid, among this polymorphism, to thereby inhibit the nucleic acid chain extension under a low-temperature environment including the annealing step, and to destabilize the hybrid between the type I detection primer and the non-type I nucleic acid. Therefore, the nonspecific-nucleic acid chain extension reaction can be efficiently inhibited, and the polymorphism identification accuracy can be improved.

Preferably, the inhibitory oligonucleotide is a kind of oligonucleotide which has no function as a primer. Here, the term “no function as a primer” means no bindability of the 3′-end nucleotide of the oligonucleotide to any new nucleotide even with the aid of a polymerase, and thus means no capability to extend a nucleic acid chain. Specifically, such a kind of oligonucleotide which has no function as a primer can be made by blocking the 3′-end nucleotide. The 3′-end nucleotide can be blocked by any known method in the art. Examples of such a blocking method can include; a method for substituting the hydroxyl group at the 3′ position of the 3′-end nucleotide of the inhibitory oligonucleotide with a functional group other than the hydroxyl group, a method for substituting the 3′-end nucleotide with a dideoxy nucleotide, and a method for binding a dye, a fluorescent molecule, a quencher molecule, an amino group, or the like to the 3′ position of the 3′-end nucleotide (via a linker, if necessary).

In the present invention, there is an overlap between the 5′ side of the region which can hybridize with the type I detection primer and the 3′ side of the region which can hybridize with the inhibitory oligonucleotide, within the type I nucleic acid. That is, the type I detection primer and the inhibitory oligonucleotide have to be respectively designed so that the 3′ side of the type I detection primer and the 5′ side of the inhibitory oligonucleotide can respectively hybridize with the same region in the type I nucleic acid. Specifically, the 3′ side of the type I detection primer and the 5′ side of the inhibitory oligonucleotide have to have a homologous nucleotide sequence, except for the polymorphism detection site to hybridize with the polymorphic site.

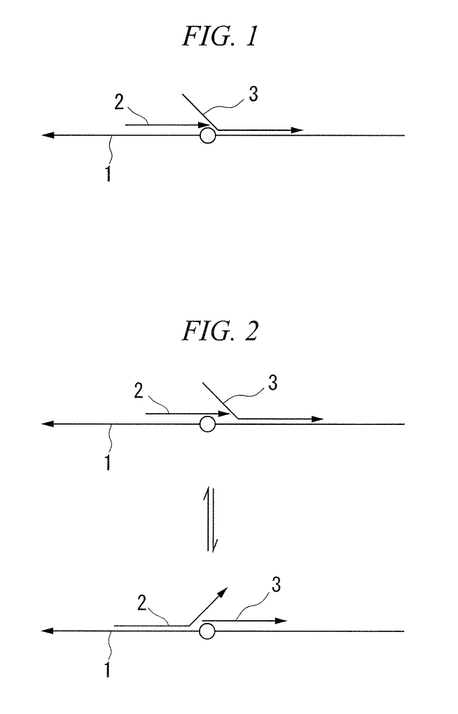

FIG. 1 is a schematic diagram showing a hybrid formed by hybridization between a type I nucleic acid (1) and a type I detection primer (2), and a hybrid formed by hybridization between the type I nucleic acid (1) and an inhibitory oligonucleotide (3). The open circle in the figure represents the polymorphic site, and the region with two parallel arrows represents a region which can hybridize with the 3′ side of the type I detection primer as well as with the 5′ side of the inhibitory oligonucleotide while both overlap with each other (hereinunder, may be simply referred to as the “overlap region”).

Since the type I nucleic acid and the type II nucleic acid are homologous except for the nucleotide sequence of the polymorphic site, the inhibitory oligonucleotide comprising a nucleotide sequence complementary to the type II nucleic acid can hybridize with the type I nucleic acid too, under a low-temperature environment, for example, in the annealing step of the nucleic acid chain extension reaction. Without the existence of the inhibitory oligonucleotide, the detection primer does elongate in the annealing step because the polymerase is active. On the other hand, with the existence of the inhibitory oligonucleotide, since the nucleic acid chain extension reaction is blocked until a temperature at which the inhibitory oligonucleotide is separated (a temperature higher than the temperature of the annealing step), it is expected that the nucleic acid chain extension reaction is limited to occur only under a high-temperature environment.

In addition, the type I detection primer and the inhibitory oligonucleotide can hybridize not only with the type I nucleic acid but also with the type II nucleic acid. FIG. 2 is a schematic diagram showing a state where the type I nucleic acid (1) hybridizes with the type I detection primer (2) and with the inhibitory oligonucleotide (3), and a state where the type II nucleic acid (4) hybridizes with the type I detection primer (2) and with the inhibitory oligonucleotide (3). The open circle in the figure and the region with two parallel arrows refer to the same meanings as those of FIG. 1. The type I nucleic acid and the type II nucleic acid have different nucleotide sequences at the polymorphic site. Therefore, as shown in FIG. 2, the end microstructures are different between the hybrid of the type I nucleic acid with the type I detection primer and with the inhibitory oligonucleotide and the hybrid of the type II nucleic acid therewith. Therefore, a variation can be made in the stability of the inhibitory oligonucleotide, by which the polymorphism identification accuracy can be expected to improve.

Moreover, the ‘branched hybridization (branch migration)’ as shown in FIG. 1 and FIG. 2 is known to be in a state where the hybridized nucleotides are at equilibrium because the junction point migration involves only a small energy transition (for example, refer to Proceedings of the National Academy of Sciences of the United States of America, 1994, Vol. 91, No. 6, pp. 2021-2025). That is, the hybrid fowled by hybridization between the type I nucleic acid and the type I detection primer, and the hybrid formed by hybridization between the type I nucleic acid and the inhibitory oligonucleotide are expected to be at equilibrium. Therefore, the polymerase for use in the nucleic acid chain extension reaction must have no strand displacement activity.

The length of the overlap region is not specifically limited as long as the length can make a difference in the equilibrium of the ‘branched hybridization’ as shown in FIG. 1 and FIG. 2 to an extent that can offer a sufficient inhibitory effect on the nonspecific-nucleic acid chain extension reaction starting from the type I detection primer. The length can be appropriately determined with consideration of the type of the nucleotide sequence of the polymorphic site, the reaction condition of the nucleic acid chain extension reaction, and the like. For example, in the present invention, the length of the overlap region is preferably five nucleotides or shorter, and more preferably two nucleotides. In addition, the nucleotide length may be equal to or shorter than twice the length of the polymorphic site. Particularly, the type I detection primer is preferably such that the 3′-end nucleotide serves as the polymorphism detection site, and a portion containing the above-mentioned 3′-end nucleotide and the second to fifth nucleotides from the 3′ end, preferably a portion up to the second nucleotide from the 3′ end, serves as the region to hybridize with the overlap region of the type I nucleic acid.

If the type I detection primer is designed such that the 3′-end nucleotide serves as the polymorphism detection site and the portion up to the second nucleotide from the 3′ end serves as the region to hybridize with the overlap region, the inhibitory oligonucleotide has to be designed such that the second nucleotide from the 5′ end serves as the polymorphism detection site and the portion up to the second nucleotide from the 5′ end serves as the region to hybridize with the overlap region. In the same manner, if the type I detection primer is designed such that the 3′-end nucleotide serves as the polymorphism detection site and the portion up to the fifth nucleotide from the 3′ end serves as the region to hybridize with the overlap region, the inhibitory oligonucleotide has to be designed such that the fifth nucleotide from the 5′ end serves as the polymorphism detection site and the portion up to the fifth nucleotide from the 5′ end serves as the region to hybridize with the overlap region.

In addition, it is preferable that the ‘branched hybridization (branch migration)’ as shown in FIG. 1 and FIG. 2 is at equilibrium under a low-temperature (annealing) condition, and that the inhibitory oligonucleotide separates from the template type I nucleic acid at the time of the nucleic acid chain extension reaction. The reason is that, by designing the inhibitory oligonucleotide in this way, it becomes possible to inhibit only the nonspecific-nucleic acid chain extension reaction without inhibiting the nucleic acid chain extension reaction of interest starting from the type I detection primer with use of the template type I nucleic acid.

Specifically, when the nucleic acid chain extension reaction comprises a denaturation step for denaturing the nucleic acid in the test nucleic acid sample into single strands, and an annealing step for hybridizing between the single-stranded nucleic acid and the type I detection primer or the inhibitory oligonucleotide; it is preferable to design the inhibitory oligonucleotide so that the Tm value of the hybrid between the type I nucleic acid and the inhibitory oligonucleotide would be higher than the temperature of the annealing step and lower than the temperature of the extension step.

In addition, it is also possible to design the type I detection primer and the inhibitory oligonucleotide so that the thermodynamic stability of the hybrid between the type I nucleic acid and the inhibitory oligonucleotide would be lower than that of the hybrid between the type I nucleic acid and the type I detection primer.

In the present invention, the Tm value of the detection primer and the inhibitory oligonucleotide can be calculated based on their nucleotide sequences by a usual method. This calculation can also be done by using a simulation software available in the market such as the Visual OMP (manufactured by DNA Software).

Specifically, in the polymorphism identification method of the present invention, first, the nucleic acid chain extension reaction is carried out by using: the nucleic acid in the test nucleic acid sample as a template, the type I detection primer, and a polymerase having no strand displacement activity, with the existence of the inhibitory oligonucleotide (elongation step).

In the nucleic acid chain extension reaction of the present invention, a polymerase having no strand displacement activity is used. As described above, the structure of the hybrid of the type I nucleic acid with the type I detection primer and with the inhibitory oligonucleotide is dynamic at equilibrium. For this reason, unlike the method described in Patent Document 2, the inhibitory effect of the inhibitory oligonucleotide on the nonspecific-nucleic acid chain extension reaction would not work if an enzyme having a strand displacement activity is used.

The nucleic acid chain extension reaction may be performed either once or a plurality of times. For example, like a PCR method, a cycle consisting of the denaturation step, the annealing step, and the extension step can be repeated twice or more times. Even if the nucleic acid chain extension reaction is performed once only, the signal for detecting the yielded nucleic acid chain extension product can be enhanced by using nucleotides labeled with a fluorophore or the like.

In addition, the nucleic acid chain extension reaction may be either a reaction like PCR which requires a polymorphism-specific primer such as the type I detection primer and a polymorphism-nonspecific primer, or a reaction like a SSPCE (Sequence-Specific Primer Cycle Elongation) method (for example, refer to Current Pharmaceutical Biotechnology, 2003, Vol. 4, pp. 477-484) which uses only the type I detection primer.

The reaction condition of the nucleic acid chain extension reaction is not specifically limited and can be appropriately determined with consideration of: the type of the polymerase for use, the Tm values of the type I detection primer and the inhibitory oligonucleotide, and the like.

In addition, the reagents such as a polymerase, nucleotides, and a buffer for use in the nucleic acid chain extension reaction are not specifically limited, and those for use in usual nucleic acid chain extension reactions can be used at usual amounts.

The nucleic acid chain extension reaction of the present invention may be performed, like multiplex PCR, additionally with the presence of a detection primer which can hybridize with a region including the polymorphic site of a nucleic acid whose polymorphic site nucleotide sequence consists of a nucleotide sequence differing from that of the first nucleotide sequence. This detection primer other than the type I detection primer is preferably the type II detection primer which can hybridize with a region including the polymorphic site of the type II nucleic acid. In addition, when the polymorphism has three types of polymorphic site nucleotide sequences, the detection primer may be a of primer which can hybridize with a nucleic acid whose polymorphic site nucleotide sequence consists of a nucleotide sequence differing from the first nucleotide sequence and the second nucleotide sequence.

The type II detection primer, similarly to the type I detection primer, may be any kind of primer which can hybridize with a partial region of the type II nucleic acid which contains the polymorphic site. The type II detection primer may be an oligonucleotide which comprises a nucleotide sequence completely complementary to the nucleotide sequence of the type II nucleic acid, or an oligonucleotide which comprises a nucleotide sequence complementary thereto except for one or several nucleotide mismatch(es). In addition, the type II detection primer may also have on its 5′ side an additional nucleotide sequence besides the region which can hybridize with the type II nucleic acid. Moreover, the type II detection primer may be labeled. Examples of the additional nucleotide sequence and the labeling substance are similar to those of the type I detection primer.

When the nucleic acid chain extension reaction of the present invention is carried out with the presence of the type II detection primer, it is preferable that the thermodynamic stability of the hybrid between the type I nucleic acid and the inhibitory oligonucleotide is lower than that of the hybrid between the type I nucleic acid and the type I detection primer, and the thermodynamic stability of the hybrid between the type II nucleic acid and the inhibitory oligonucleotide is lower than that of the hybrid between the type II nucleic acid and the type II detection primer. By respectively setting the thermodynamic stabilities of the hybrids between the inhibitory oligonucleotide and the nucleic acids lower than the thermodynamic stabilities of the hybrids between these detection primers and the corresponding nucleic acids, the influence of the inhibitory oligonucleotide on the nucleic acid chain extension reaction of each detection primer can be reduced.

The type I detection primer, the type II detection primer, and the inhibitory oligonucleotide for use in the present invention can be designed by any method well known in the art, according to the nucleotide sequence of the identification target polymorphic site and the vicinity thereof. For example, these can be easily designed by using publicly known genome sequence data or SNP data with a general primer design tool. The publicly known genome sequence data is usually available on international nucleotide sequence databases, namely NCBI (National Center for Biotechnology Information), DDBJ (DNA Data Bank of Japan), and the like. Examples of the primer design tool include Primer3 (Rozen, S., H. J. Skaletsky, 1996, http://www-genome.wi.mitedu/genome_software/other/primer3.html) and Visual OMP (DNA Software) which are available on the web.

The thus designed primers and the like can be synthesized by any method well known in the art. For example, they may be synthesized by a custom oligo synthesis service, or may be synthesized by a user themselves using a commercially available synthesizer.

Next, the polymorphism of the nucleic acid contained in the test nucleic acid sample is identified depending on whether or not the type I detection primer has been extended in the extension step (identification step). That is, if a nucleic acid chain extension product starting from the type I detection primer is detected, the test nucleic acid sample can be determined to contain the type I nucleic acid.

For example, when the identification target polymorphism is a SNP having two types of polymorphism, namely a wild-type and a mutant, and when the nucleotide sequence of the mutant is regarded as the first nucleotide sequence and the nucleotide sequence of the wild-type is regarded as the second nucleotide sequence, then if a nucleic acid chain extension product of the type I detection primer is detected, the nucleic acid in the test nucleic acid sample can be determined to contain the mutant allele, and the donor of the test nucleic acid sample can be determined to have a homozygote or heterozygote of the mutant allele. On the other hand, when the identification target polymorphism is a somatic mutation, and when the nucleotide sequence of the mutant is regarded as the first nucleotide sequence and the nucleotide sequence of the normal type is regarded as the second nucleotide sequence, then if the nucleic acid chain extension product of the type I detection primer is detected, the nucleic acid in the test nucleic acid sample can be determined to contain the mutant nucleic acid, and the donor of the test nucleic acid sample can be determined to experience the somatic mutation.

The detection method of the nucleic acid chain extension product in the identification step is not specifically limited, and can be appropriately selected from known methods for use in quantitative measurements of nucleic acid chain extension products. For example, the detection may be done by electrophoresis or column chromatography based on the difference in the nucleotide length, or may be done by TOF-MS or such mass spectrometry.

When the detection primer is pre-labeled with a labeling substance, the detection can be done by the signal indication from the labeling substance. For example, when the detection primer is labeled with a fluorophore, the ratio of the amount of nucleic acid strand extension product to the amount of the unreacted primer can be measured by any one or more methods selected from the group consisting of Fluorescence Correlation Spectroscopy (hereinunder, referred to as FCS), Fluorescence Intensity Distribution Analysis (hereinunder, referred to as FIDA), and FIDA-polarization (hereinunder, referred to as FIDA-PO). Then, based on this ratio of the amount of nucleic acid strand extension product to the amount of the unreacted primer, the nucleic acid chain extension product can be detected.

Furthermore, if the type I detection primer and the inhibitory oligonucleotide used in the polymorphism identification method of the present invention are prepared as a kit set, the polymorphism identification method of the present invention can be more easily performed. In addition, the kit may also include an enzyme for use in the nucleic acid chain extension reaction, a buffer for preparing the reaction solution, nucleotides, and other reagents.

### EXAMPLES

Next is a more detailed description of the present invention with reference to examples. However, the present invention is not to be considered as being limited by these examples.

### Example 1

The polymorphism identification accuracy of the polymorphism identification method of the present invention was verified by SSP-PCR with or without the presence of the inhibitory oligonucleotide.

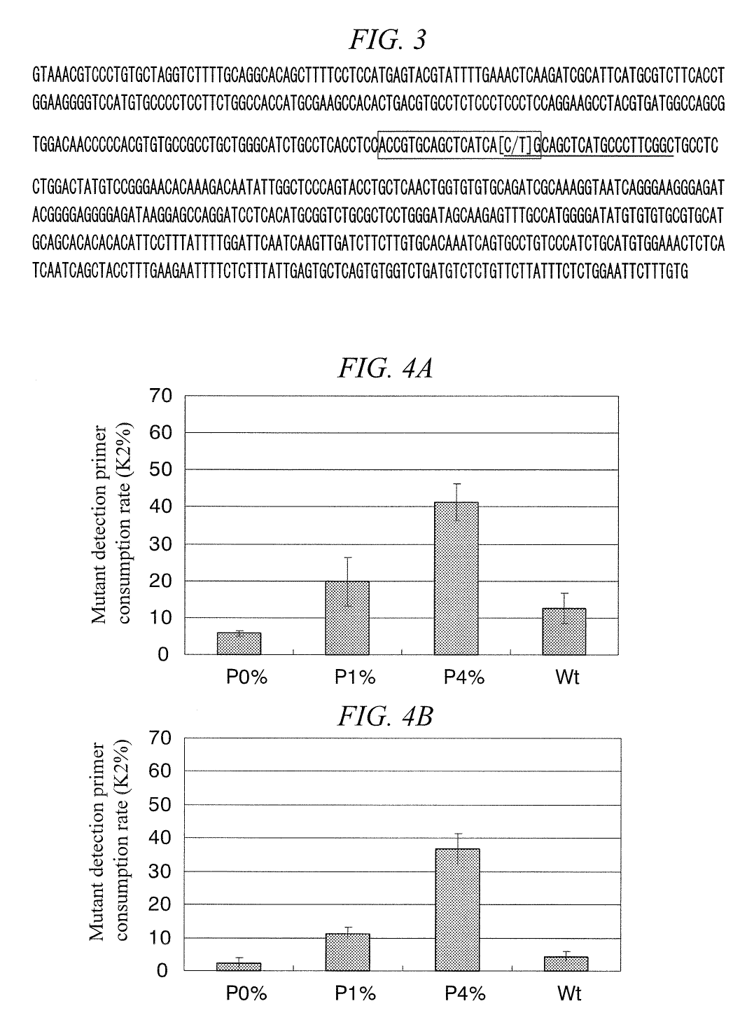

Specifically, the identification accuracy was investigated for the EGFR (epidermal growth factor receptor) gene T790M mutation which is quite frequently found in tumor cells. This somatic mutation is a kind of mutation in which threonine-to-methionine substitution at the amino acid position 790 in EGFR occurs due to C-to-T substitution at the second nucleotide C in the threonine-encoding codon ACG FIG. 3 shows the nucleotide sequence in the vicinity of the threonine-encoding codon at the amino acid position 790 in the EGFR gene, wherein [C/T] represents the polymorphic site (T790M polymorphic site). The nucleotide sequence with C at the polymorphic site was regarded as the nucleotide sequence of the wild-type (second nucleotide sequence), and the nucleotide sequence with T at the polymorphic site was regarded as the nucleotide sequence of the mutant (first nucleotide sequence).

### Production of Detection Primer and Inhibitory Oligonucleotide

The mutant detection primer was designed and produced so as to have the polymorphism detection site at the 3′-end nucleotide of the primer and to hybridize with a region including the T790M polymorphic site of the mutant nucleic acid as shown in FIG. 3. The 5′-end nucleotide of the mutant detection primer was conjugated with the ATTO647N fluorophore (manufactured by ATTO-TEC GmbH) to label the primer with fluorescence (manufactured by SIGMA Genosys, HPLC grade) (SEQ. ID. NO. 1).

Moreover, the wild-type detection primer was designed and produced by having an A-to-G substitution at the polymorphism detection site (nucleotide at the 3′ end) in the mutant detection primer. The 5′-end nucleotide of the wild-type detection primer was conjugated with the TAMRA fluorophore (manufactured by SIGMA Genosys, HPLC grade) (SEQ. ID. NO. 2).

Meanwhile, the inhibitory oligonucleotide was designed and produced so as to have a nucleotide sequence complementary to a region including the T790M polymorphic site of the wild-type nucleotide sequence, and to overlap with the 3′ end of the mutant detection primer (or the wild-type detection primer) by two nucleotides, when hybridizing with the mutant nucleic acid (or the wild-type nucleic acid). Furthermore, the hydroxyl group of the 3′-end nucleotide was modified with an amino group to hinder the function as a primer (manufactured by SIGMA Genosys, cartridge purification) (SEQ. ID. NO. 3).

In FIG. 3, the underlined region represents a region where the hybridization occurs between the mutant/wild-type detection primer and the mutant/wild-type nucleic acid. Moreover, in FIG. 3, the framed region represents a region where the hybridization occurs between the inhibitory oligonucleotide and the mutant/wild-type nucleic acid. The 5′ side of the underlined region and the 3′ side of the framed region overlap with each other by two nucleotides including the T790M polymorphic site.

TABLE 1

| | Nucleotide sequence |
|---|---|
| SSP-Primer T | ATTO-GCCGAAGGCATGAGCTGCA |
| SSP-Primer C | TAMRA-GCCGAAGGGCATGAGCTGCG |
| PCR Optimizer | CGTGATGAGCTGCACGGT-NH₂ |

TABLE 2

Thermodynamic stability
(Tm value)

| | Mutant Type | Wild Type |
|---|---|---|
| SSP-Primer T | 56.7° C. | 60.4° C. |
| SSP-Primer C | 63.3° C. | 63.0° C. |
| PCR Optimizer | 63.7° C. | 68.8° C. |

Table 1 shows the nucleotide sequences of the mutant detection primer, the wild-type detection primer, and the inhibitory oligonucleotide. In the table, the “SSP-Primer T” represents the mutant detection primer, the “SSP-Primer C” represents the wild-type detection primer, and the “PCR Optimizer” represents the inhibitory oligonucleotide.

In addition, Table 2 shows the thermodynamic stabilities, in terms of the Tm value, of the hybrids formed by the hybridizations of the mutant detection primer, the wild-type detection primer, and the inhibitory oligonucleotide, to the mutant nucleic acid and the wild-type nucleic acid. The Tm value was obtained by calculation using the simulation software Visual OMP (manufactured by DNA Software), under the condition of 50 mM Na⁺, 2.5 mM MgCl₂, [detection primer or inhibitory oligonucleotide]=100 nM, and [mutant nucleic acid or wild-type nucleic acid]=1 nM.

As is apparent from Table 2, the inhibitory oligonucleotide was designed so that the Tm value of the hybrid between the inhibitory oligonucleotide and the mutant/wild-type nucleic acid would be higher than the temperature of the annealing step (60° C.) and lower than the temperature of the extension step (72° C.) in the following SSP-PCR. In this way, by designing the inhibitory oligonucleotide so that it can reliably bind to the respective template nucleic acids in the annealing step and can be separated from the respective template nucleic acids in the extension step, it becomes possible to efficiently inhibit the nonspecific-nucleic acid chain extensions without excessively inhibiting the nucleic acid chain extension of interest.

Preparation of Standard Test Nucleic Acid Sample

In order to verify the identification accuracy for the T790M polymorphism by using the polymorphism identification method of the present invention, standard test nucleic acid samples containing the mutant nucleic acid and the wild-type nucleic acid at known content ratios were prepared.

First, the mutant nucleic acid and the wild-type nucleic acid were mixed so that the content ratio of the mutant nucleic acid (the ratio of the mutant nucleic acid relative to the total amount of the mutant nucleic acid and the wild-type nucleic acid) would be respectively 0%, 1%, 4%, 50%, and 100%, to thereby prepare a five-types concentration series of the standard test nucleic acid sample with known content ratios of the mutant nucleic acid. The mutant nucleic acid and the wild-type nucleic acid used for the preparation of the standard test nucleic acid sample were obtained after PCR amplification using two types of primers shown in Table 3 (1st PCR-Primer 1 and 1st PCR-Primer 2, both manufactured by SIGMA Genosys, desalination grade) (SEQ. ID. NOs. 4, and 5) with the template nucleotide sequences shown in FIG. 3, and introduction into plasmids upon confirmation of the nucleotide sequences of the resultant amplification products.

TABLE 3

| | Nucleotide sequence |
|---|---|
| 1stPCR Primer1 | CACAAAGAATTCCAGAGAAATA |
| 1stPCR-Primer2 | GTAAACGGTCCCTGTGCT |

### Healthy Sample

Fifty types of genome samples purchased from the Japan Health Sciences Foundation were used as healthy samples. In the following PCR, each genome sample was diluted at 20 ng/4 with TE Buffer (10 mM Tris-HCl, 1 mM EDTA) for use as a genome sample solution.

### First Round PCR

In usual genetic tests, genomic fragments including the polymorphic site are pre-amplified so as to obtain a sufficient amount of template, and the thus obtained amplification product is used as a template to perform the nucleic acid chain extension reaction such as SSP-PCR for polymorphism identification.

Similarly, in this example, in order to obtain sufficient amounts of templates, the mutant nucleic acid and the wild-type nucleic acid were re-amplified by PCR using the prepared standard test nucleic acid sample or the genome sample as a template with the 1st PCR-Primer 1 and the 1st PCR-Primer 2. Since the same primers were used, this pre-amplification treatment would not affect the content ratio of the mutant nucleic acid in each sample.

Specifically, 1 μL of the standard test nucleic acid sample or the genome sample solution was respectively added to 10 μL, of 2× AmpliTaq Gold Master Mix (manufactured by ABI). The 1st PCR-Primer 1 and the 1st PCR-Primer 2 were added thereto at each final concentration of 0.1 μM, respectively. The resultant product was adjusted with pure water at the final volume of 20 μL, which was used as the reaction solution. This reaction solution was subjected to PCR amplification under the reaction condition consisting of; a treatment at 95° C. for 10 minutes, then 40 cycles at 95° C. for 30 seconds, 65° C. for 30 seconds, and 72° C. for 30 seconds, and an additional treatment at 72° C. for 10 minutes. The resulting PCR-reacted solution was used as the test nucleic acid sample after the pre-amplification treatment (pre-amplified test nucleic acid sample).

### Second Round PCR (SSP-PCR)

The pre-amplified test nucleic acid sample after the pre-amplification treatment through the first round PCR was used as a template, and subjected to SSP-PCR with or without the presence of the inhibitory oligonucleotide. Titanium Taq (manufactured by TaKaRa) having no strand displacement activity was used as a DNA polymerase.

Specifically, in the reaction with the presence of the inhibitory oligonucleotide, 1 μL of each pre-amplified test nucleic acid sample was added to 2 μL of 10× Titanium Taq Buffer (manufactured by TaKaRa). The mutant detection primer, the wild-type detection primer, and the inhibitory oligonucleotide were added thereto at each final concentration of 0.01 μM, respectively. The resultant product was further added with 1.6 μL of dNTP Blend (10 mM, manufactured by TaKaRa) and 0.1 μL of 50× Titanium Taq (manufactured by TaKaRa). The mixture was adjusted with pure water at the final volume of 20 μL, which was used as the reaction solution. These reaction solutions were subjected to SSP-PCR under the reaction condition consisting of; a treatment at 95° C. for 2 minutes, then 40 cycles at 95° C. for 30 seconds, 60° C. for 30 seconds, and 72° C. for 30 seconds, and an additional treatment at 72° C. for 10 minutes.

On the other hand, regarding the reaction solution, a solution containing not the inhibitory oligonucleotide but an equal amount of pure water instead was prepared, and used as the reaction solution for the reaction without the presence of the inhibitory oligonucleotide. These reaction solutions were subjected to SSP-PCR under the reaction condition consisting of; a treatment at 95° C. for 2 minutes, then 40 cycles at 95° C. for 30 seconds, 65° C. for 30 seconds, and 72° C. for 30 seconds, and an additional treatment at 72° C. for 10 minutes. That is, in the reaction without the presence of the inhibitory oligonucleotide, the annealing temperature was set 5° C. higher than that of the reaction with the presence of the inhibitory oligonucleotide.

### Measurement of Primer Consumption Rate

The PCR products resulting from the second round PCR were diluted 10-fold with 10 mM Tris-HCl, and measured for the primer consumption rates (K2%) of the mutant detection primer and the wild-type detection primer after the second round PCR respectively by the Fluorescence Correlation Spectroscopy (hereinunder, referred to as FCS).

Here, the primer consumption rate is a value calculated by the following equation.

Primer consumption rate=[amount of nucleic acid strand extension product]/[initial primer amount]=[amount of nucleic acid strand extension product]/([amount of nucleic acid strand extension product]+[amount of unreacted primer])

FCS measurement was carried out with the fluorescence correlation spectrometer MF-20 (manufactured by Olympus). The measurement was for 15 seconds three times per each sample, and the average value thereof was used as the measurement result. Of the components resulting from the measurement, a component exhibiting a short diffusion time was assumed to be the unreacted primer and a component exhibiting a long diffusion time was assumed to be the nucleic acid chain extension product, by which the ratio of them was obtained. Then, based on this ratio, the primer consumption rate (K2%) was calculated.

FIG. 4 shows the measured consumption rate (K2%) of the mutant detection primer with variations of the content ratio of the mutant nucleic acid. FIG. 4A shows the result of the reaction with the presence of the inhibitory oligonucleotide, and FIG. 4B shows the result of the reaction without the presence of the inhibitory oligonucleotide. In these graphs, the term “Pn %” means that the content ratio of the mutant nucleic acid in the standard test nucleic acid sample used as the template is n %. In addition, the terra “Wt” means the result of the genome sample solutions used as the templates (the average of fifty types).

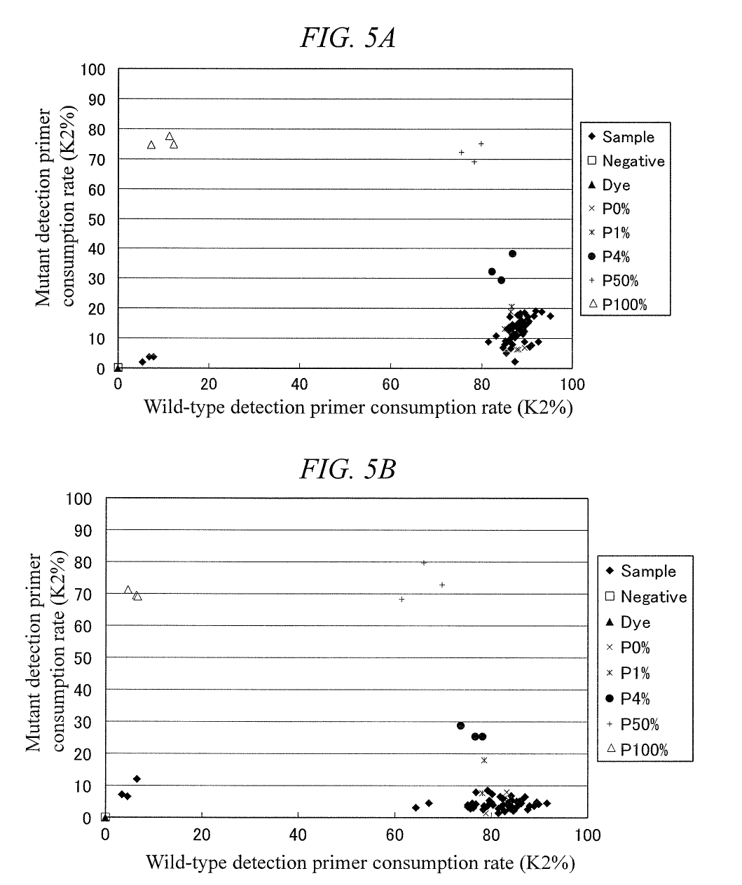

Moreover, FIG. 5 shows the result of the FCS measurement for respective test nucleic acid samples in scatter charts, wherein the y axis represents the consumption rate (K2%) of the mutant detection primer and the x axis represents the consumption rate (K2%) of the wild-type detection primer. FIG. 5A shows the result of the reaction with the presence of the inhibitory oligonucleotide, and FIG. 5B shows the result of the reaction without the presence of the inhibitory oligonucleotide. In these charts, the open square shows the result of the negative control containing pure water instead of the template in the first PCR (Negative), the solid triangle shows the result of a sample containing nothing but the fluorophore in the FCS measurement (Dye), and the solid diamond shows the result of the genome sample solution (Sample). In addition, the letter x, the asterisk, the solid circle, the letter +, and the open triangle respectively show the results of P0%, P1%, P4%, P50%, and P100%.

As a result, when the content ratio of the mutant nucleic acid was 0% or 1% in the template (P0% or P1%), the primer consumption rate of the mutant detection primer was significantly lower with the presence of the inhibitory oligonucleotide than without the presence of the inhibitory oligonucleotide. On the other hand, when the content ratio of the mutant nucleic acid was 4% (P4%), there was no big difference between with and without the presence of the inhibitory oligonucleotide, although the case with the presence of the inhibitory oligonucleotide showed a slightly lower value.

Ideally, when the content ratio of the mutant nucleic acid is 0% in the template, the primer consumption rate of the mutant detection primer will be 0%. That is, these results confirmed that the nonspecific-nucleic acid chain extension reactions were efficiently inhibited by adding the inhibitory oligonucleotide to the reaction solution of the nucleic acid chain extension reaction.

Also, regarding the healthy samples (fifty types of genome sample solutions), the primer consumption rate of the mutant detection primer was significantly lower with the presence of the inhibitory oligonucleotide than without the presence of the inhibitory oligonucleotide. In addition, the variability (standard deviation) of the measurement results of the fifty samples was 4% without the presence of the inhibitory oligonucleotide and 1.52% with the presence of the inhibitory oligonucleotide, which confirmed that the variability decreased by adding the inhibitory oligonucleotide. This can be attributed to the inhibition on nonspecific-nucleic acid chain extension reactions by adding the inhibitory oligonucleotide.

In this manner, by adding the inhibitory oligonucleotide, the signal of nonspecific-nucleic acid chain extension reaction decreased in the healthy samples and the annealing temperature in SSP-PCR also decreased. Therefore, the polymorphism identification method of the present invention using the inhibitory oligonucleotide has a higher robustness than prior art methods.

### Example 2

In the reaction system containing the inhibitory oligonucleotide, the polymorphism identification accuracy was compared between the case using a polymerase having a strand displacement activity and the case using a polymerase having no strand displacement activity.

The identification accuracy for the polymorphism of EGFR T790M was verified by using the mutant detection primer, the wild-type detection primer, and the inhibitory oligonucleotide used in the example 1, with the respective samples in the concentration series of the standard test nucleic acid sample that had been prepared in the “Preparation of standard test nucleic acid sample” of the example 1 as the test nucleic acid sample.

### First Round PCR

First, 1 μL of the standard test nucleic acid sample was added to 10 μL of 2× AmpliTaq Gold Master Mix (manufactured by ABI). The 1st PCR-Primer 1 and the 1st PCR-Primer 2 used in the example 1 were added thereto at each final concentration of 0.1 μM, respectively. The resultant product was adjusted with pure water at the final volume of 20 μL, which was used as the reaction solution. This reaction solution was subjected to PCR amplification under the reaction condition consisting of a treatment at 95° C. for 10 minutes, then 40 cycles at 95° C. for 30 seconds, 65° C. for 30 seconds, and 72° C. for 30 seconds, and an additional treatment at 72° C. for 10 minutes. The resulting PCR-reacted solution was used as the test nucleic acid sample after the pre-amplification treatment (pre-amplified test nucleic acid sample).

### Second Round PCR (SSP-PCR)

The pre-amplified test nucleic acid sample after the pre-amplification treatment through the first round PCR was used as a template, and subjected to SSP-PCR with or without the presence of the inhibitory oligonucleotide. Deep Vent (exo⁻) DNA polymerase (manufactured by NEB) was used as a polymerase having a strand displacement activity, and Titanium Taq (manufactured by TaKaRa) of the example 1 was used as a polymerase having no strand displacement activity.

The composition of the reaction solution for the reaction with the polymerase having no strand displacement activity was set the same as that of the example 1.

Meanwhile, the composition of the reaction solution for the reaction with the polymerase having a strand displacement activity was set the same as that of the reaction solution for the reaction with the polymerase having no strand displacement activity, except that 10× TermoPol Reaction Buffer (manufactured by NEB) was used instead of 10× Titanium Taq Buffer, and Deep Vent (exo⁻) DNA polymerase (manufactured by NEB, 2000 U/mL) was used instead of 50× Titanium Taq (manufactured by TaKaRa).

These reaction solutions were subjected to SSP-PCR under the reaction condition consisting of a treatment at 95° C. for 2 minutes, then 40 cycles at 95° C. for 30 seconds, 66° C. for 30 seconds, and 72° C. for 30 seconds, and an additional treatment at 72° C. for 10 minutes.

### Measurement of Primer Consumption Rate

The PCR products resulting from the second round PCR were subjected to the FCS measurement in the same manner as that of the example 1, to measure the primer consumption rates (K2%) of the mutant detection primer and the wild-type detection primer after the second round PCR respectively.

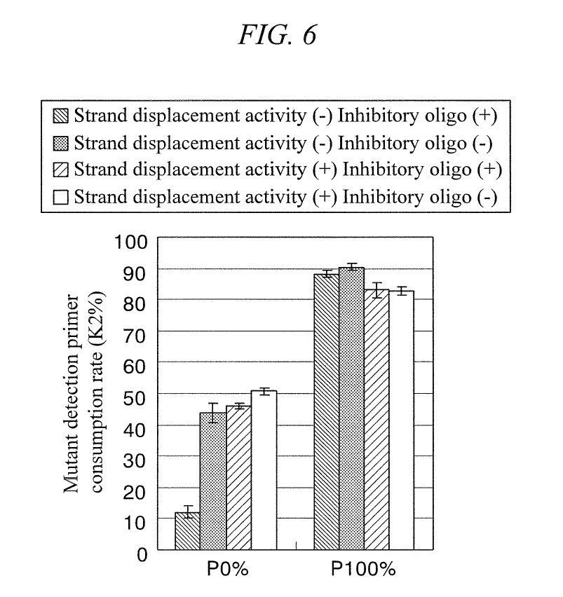

FIG. 6 shows the measured consumption rate (K2%) of the mutant detection primer with variations of the content ratio of the mutant nucleic acid. In the graph, the term “P0%” shows the results of the standard test nucleic acid sample having 0% content ratio of the mutant nucleic acid as a template (having only the wild-type nucleic acid as a template), and the term “P100%” shows the results of the standard test nucleic acid sample having 100% content ratio of the mutant nucleic acid as a template (having only the mutant nucleic acid as a template). In the graph, the term “Strand displacement activity (−) Inhibitory oligo (+)” shows the results with the polymerase having no strand displacement activity and with the inhibitory oligonucleotide, the term “Strand displacement activity (−) Inhibitory oligo (−)” shows the results with the polymerase having no strand displacement activity and without the inhibitory oligonucleotide, the term “Strand displacement activity (+) Inhibitory oligo (+)” shows the results with the polymerase having a strand displacement activity and with the inhibitory oligonucleotide, and the term “Strand displacement activity (+) Inhibitory oligo (−)” shows the results with the polymerase having a strand displacement activity and without the inhibitory oligonucleotide.

As a result, when only the wild-type nucleic acid was used as a template (P0%), with the polymerase having a strand displacement activity (Deep Vent (exo⁻) DNA polymerase), a signal showing a large molecular weight was detected in the FCS measurement irrespective of the addition of the inhibitory oligonucleotide, and their consumption rates of the mutant detection primer were 40 to 50%, confirming the production of the nucleic acid chain extension reaction product. On the other hand, with use of the polymerase having no strand displacement activity (Titanium Taq), the consumption rate of the mutant detection primer without the addition of the inhibitory oligonucleotide was still high similarly to the case with the polymerase having a strand displacement activity; however, the consumption rate of the mutant detection primer with the addition of the inhibitory oligonucleotide was very low at about 10%.

On the other hand, when only the mutant nucleic acid was used as a template (P100%), the consumption rate of the mutant detection primer was found to be higher with the polymerase having no strand displacement activity than with the polymerase having a strand displacement activity. Moreover, there was no big difference found between with and without the addition of the inhibitory oligonucleotide.

That is, when only the wild-type nucleic acid was used as a template (P0%), the only case which exhibited sufficient inhibition on nonspecific-nucleic acid chain extension reactions and was capable of detecting the polymorphism was the case where the inhibitory oligonucleotide was added and the polymerase having no strand displacement activity was used (that is, the case where the polymorphism identification method the present invention was employed).

### INDUSTRIAL APPLICABILITY

The polymorphism detection method of the present invention is capable of satisfactory detection of a somatic mutation or such a polymorphism which requires high detection sensitivity, and thus is useful particularly in the field of genetic tests including SNP identification as well as clinical tests including analyses on tumor-related somatic mutations.

### BRIEF DESCRIPTION OF THE REFERENCE SYMBOLS

1: Type I nucleic acid, 2: Type I detection primer, 3: Inhibitory oligonucleotide, 4: Type II nucleic acid
Sequence Listing

What is claimed is:

1. A polymorphism identification method for identifying a polymorphism of a polymorphic site-containing nucleic acid, comprising:
(a) performing a nucleic acid chain extension reaction with use of a nucleic acid in a test nucleic acid sample as a template, a type I detection primer, and a polymerase having no strand displacement activity, with the presence of an inhibitory oligonucleotide, the type I detection primer being a primer which can hybridize with a nucleic acid in a region including the polymorphic site thereof whose polymorphic site nucleotide sequence consisting of a first nucleotide sequence, and the inhibitory oligonucleotide being an oligonucleotide which comprises a nucleotide sequence complementary to the sequence of a region including the polymorphic site of a nucleic acid whose polymorphic site nucleotide sequence consisting of a second nucleotide sequence; and
(b) identifying the polymorphism of the nucleic acid contained in the test nucleic acid sample, based on whether or not the type I detection primer has been extended in step (a);
wherein there is an overlap between the 5′ side of the region which can hybridize with the type I detection primer and the 3′ side of the region which can hybridize with the inhibitory oligonucleotide, within the nucleic acid whose polymorphic site nucleotide sequence consists of the first nucleotide sequence.

2. The polymorphism identification method according to claim 1, wherein the 3′-end nucleotide of the inhibitory oligonucleotide is blocked so that the oligonucleotide has no function as a primer.

3. The polymorphism identification method according to claim 1, wherein the length of the overlap region between the 5′ side of the region which can hybridize with the type I detection primer and the 3′ side of the region which can hybridize with the inhibitory oligonucleotide, within the nucleic acid whose polymorphic site nucleotide sequence consists of the first nucleotide sequence, is five nucleotides or less.

4. The polymorphism identification method according to claim 1, wherein the length of the overlap region between the 5′ side of the region which can hybridize with the type I detection primer and the 3′ side of the region which can hybridize with the inhibitory oligonucleotide, within the nucleic acid whose polymorphic site nucleotide sequence consists of the first nucleotide sequence, is equal to or shorter than twice the length of the polymorphic site.

5. The polymorphism identification method according to claim 1, wherein the length of the overlap region between the 5′ side of the region which can hybridize with the type I detection primer and the 3′ side of the region which can hybridize with the inhibitory oligonucleotide, within the nucleic acid whose polymorphic site nucleotide sequence consists of the first nucleotide sequence, is two nucleotides or less.

6. The polymorphism identification method according to claim 1, wherein the polymorphism detection site of the type I detection primer to hybridize with the polymorphic site is located at its 3′ end.

7. The polymorphism identification method according to claim 1, wherein the nucleic acid chain extension reaction comprises
(i) denaturing the nucleic acid in the test nucleic acid sample into single strands;
(ii) annealing the single-stranded nucleic acid with the type I detection primer and/or the inhibitory oligonucleotide; and
(iii) extending the nucleic acid strand starting from the type I detection primer, wherein a Tm value of a hybrid between the inhibitory oligonucleotide and the nucleic acid whose polymorphic site nucleotide sequence consisting of the first nucleotide sequence, being higher than the temperature of (ii), and lower than the temperature of step (a).

8. The polymorphism identification method according to claim 1, wherein a thermodynamic stability of a hybrid between the inhibitory oligonucleotide and the nucleic acid whose polymorphic site nucleotide sequence consists of the first nucleotide sequence, is lower than that of a hybrid between the type I detection primer and the nucleic acid.

9. The polymorphism identification method according to claim 7, wherein a cycle consisting of (i), (ii), and (iii) is repeated twice or more times in the nucleic acid chain extension reaction.

10. The polymorphism identification method according to claim 1, wherein the nucleic acid chain extension reaction is performed on a nucleic acid whose polymorphic site nucleotide sequence is different from the first nucleotide sequence, with the presence of a detection primer which can hybridize with the nucleic acid in a region including the polymorphic site thereof.

11. The polymorphism identification method according to claim 10, wherein the detection primer is a type II detection primer which can hybridize with a nucleic acid in a region including the polymorphic site thereof whose polymorphic site nucleotide sequence consists of the second nucleotide sequence.

12. The polymorphism identification method according to claim 11, wherein a thermodynamic stability of a hybrid between the inhibitory oligonucleotide and the nucleic acid whose polymorphic site nucleotide sequence consists of the first nucleotide sequence, is lower than that of a hybrid between the type I detection primer and the nucleic acid whose polymorphic site nucleotide sequence consists of the first nucleotide sequence, and a thermodynamic stability of a hybrid between the inhibitory oligonucleotide and the nucleic acid whose polymorphic site nucleotide sequence consists of the second nucleotide sequence, is lower than that of a hybrid between the type II detection primer and the nucleic acid whose polymorphic site nucleotide sequence consists of the second nucleotide sequence.

13. A polymorphism identification kit for use in the identification of a polymorphism of a polymorphic site-containing nucleic acid, comprising:
a type I detection primer which can hybridize with a nucleic acid in a region including the polymorphic site thereof whose nucleotide sequence consisting of a first nucleotide sequence; and
an inhibitory oligonucleotide which comprises a nucleotide sequence complementary to the sequence of a region including the polymorphic site of a nucleic acid whose polymorphic site nucleotide sequence consisting of a second nucleotide sequence,
wherein there is an overlap between the 5′ side of the region which can hybridize with the type I detection primer and the 3′ side of the region which can hybridize with the inhibitory oligonucleotide, within the nucleic acid whose polymorphic site nucleotide sequence consists of the first nucleotide sequence.
